(19) United States
(12) Patent Application Publication
(10) Pub. No.: US 2020/0069725 A1
(43) Pub. Date: Mar. 5, 2020

(54) HYALURONAN COMPOSITIONS, AND USES THEREOF IN TREATMENT OF INTERSTITIAL CYSTITIS

(71) Applicant: NATIONAL UNIVERSITY OF IRELAND, GALWAY, Galway (IE)

(72) Inventors: Abhay PANDIT, Galway (IE); Vijaya KRISHNA, Galway (IE); Sit ISMAIL, Galway (IE); Peadar ROONEY, Galway (IE)

(73) Assignee: NATIONAL UNIVERSITY OF IRELAND, GALWAY, Galway (IE)

(21) Appl. No.: 16/339,512

(22) PCT Filed: Oct. 3, 2017
(86) PCT No.: PCT/EP2017/075040

§ 371 Date: Apr. 4, 2019

(30) Foreign Application Priority Data
Oct. 4, 2016 (GB) 1616838.7

(51) Int. Cl. A61K 31/728; A61K 47/26; A61K 9/00; A61K 9/06; A61P 13/10

(52) U.S. Cl. CPC A61K 31/728; A61K 47/26; A61P 13/10; A61K 9/06; A61K 9/0034

(57) ABSTRACT

A hydrogel composition for treatment of interstitial cystitis is described and comprises crosslinked hyaluronan particles dispersed throughout a crosslinked hyaluronan hydrogel matrix, wherein the hyaluronan is high molecular weight hyaluronan. The composition is formulated for direct bladder instillation in a human. The hyaluronan particles are nano-sized hyaluronan particles, or agglomerates of nano-sized hyaluronan particles, or a mixture thereof.

### FIELD OF THE INVENTION

The present invention relates to hyaluronan compositions and uses thereof to treat medical indications, especially interstitial cystitis. Also contemplated are methods of making hyaluronan compositions.

### BACKGROUND TO THE INVENTION

Interstitial cystitis/painful bladder syndrome (IC/PBS) is a chronic inflammatory disease characterised by urinary bladder pain, urinary frequency, urgency, nocturia and chronic pelvic pain, which severely affects patient quality of life. The quality of life for IC patients is rated similar to end stage renal disease or severe rheumatoid arthritis. Limitations of existing intravesical hyaluronan solution instillation therapies, the current goal standard in the clinic, lie in the inability to ensure complete recovery of the bladder mucosal barrier function. In 2011 the RAND Interstitial Cystitis Epidemiology survey estimated that 2.7% to 6.5% of United States women have urinary symptoms consistent with a diagnosis of interstitial cystitis/bladder pain syndrome. It is estimated that approximately 83.4 million women suffer from bladder disorders in the seven major markets. Presently, those products that have been approved for bladder disorders are based on clinical studies which have shown the drugs to be marginally effective.

Presently existing products that have been approved for the treatment of bladder disorders are based on clinical studies which have shown the drugs to be marginally effective. Several current therapeutic strategies are merely symptom management in nature which failing to address the underlying disease pathology. Ibuprofen, naproxen and other nonsteroidal anti-inflammatory drugs target pain symptoms. Tricyclic antidepressants, such as amitriptyline or imipramine aim to relax your bladder muscles and block pain. Antihistamines, such as diphenhydramine target mast cells activation in the bladder cell wall. All the above are oral treatments that lack clinical effectiveness. Treatment options are weak and include pentosan polysulphate sodium (Elmiron) and dimethyl sulfoxide (Rimso-50), both of which are now off patent. PPS is an oral GAG replacement treatment; studies have shown minimal therapeutic effect for patients, with only between 6.2%-18.7% of IC patients benefitting from PPS, generic PPS is not yet available. The generic dimethyl sulfoxide was launched in 2002; it is an instillation treatment, with limited clinical efficacy and a lack of clinical data, with the latest trial in 2000 showing limited effect for a subtype of IC. Gepan®, and Uracyst® chondroitin sulphate based treatments have a lack of efficacy, with chondroitin based clinical studies having no effect compared to placebo in two double, blinded, multicentre, randomised, parallel group studies.

US2010/028435 discloses injectable hyaluronan hydrogels for therapeutic and cosmetic/dermatological applications. The hydrogel comprises a hyaluronan gel matrix and relatively large (1-20 microns) crosslinked hyaluronan particles co-crosslinked to the continuous phase gel matrix. The composition is suggested for use in the urology/gynaecological field as an agent to increase the volume of a sphincter muscle.

EP2011816 describes a hyaluronan composition having a two gel system, for use as a tissue filler in cosmetic or surgical applications. The gel includes hyaluronan particles co-crosslinked with the continuous gel matrix. The gel may be administered by periurethral injection for the treatment of urinary incontinence.

US2016/038643 describes a tissue replacement scaffold comprising low molecular weight HA particles in a methacrylated HA gel matrix. The scaffold may be administered as a non-crosslinked precursor composition comprising HA particles in a HA gel matrix, where the precursor composition is activated in-vivo by means of photo-crosslinking.

WO2009/018076 describes a crosslinked high molecular weight HA gel for use as a dermal filler agent. The gel may include particles of crosslinked HA. The crosslinking agent is a multifunctional crosslinker, such as a 4-arm functionalised PEG moiety, which has been found to increase the mechanical strength of the gel to make it suitable for use as a dermal filler agent. This would make the gel unsuitable for direct bladder instillation.

It is an object of the invention to overcome at least one of the above-referenced problems.

### SUMMARY OF THE INVENTION

The present invention is based on the finding that instillation treatments of interstitial cystitis (IC) employing high molecular weight hyaluronan compositions can be improved by providing the composition in the form of hyaluronan particles dispersed throughout a gel matrix, typically a high molecular weight hyaluronan gel matrix, although other gel matrices may be employed such as alginate. The Applicant has shown that hyaluronan (HA) particles significantly increase endogenous sGAG expression compared with a range of prior art compositions in a cell model of urothelial cell inflammation (FIG. 2), and demonstrated that provision of HA particles in a HA hydrogel matrix provides a significant and consistent increase in sGAG expression in urothelial cells across a range of concentrations (FIG. 5). The Applicant has also shown that urothelial tissue explants treated with HA particles retain greater tissue integrity and thicker urothelium compared with both untreated and protamine sulphate treated tissue (FIG. 6). The Applicant has also shown that urothelial tissue explants treated with HA particles in HA gel decrease bladder permeability compared with both untreated and protamine sulphate treated tissue (FIG. 11).

Broadly, the invention relates to hyaluronan particles, typically nano-sized particles, for use in treating interstitial cystitis or other inflammatory conditions or diseases. The particles are generally provided in the form of a composition comprising the particles dispersed within a carrier phase. The carrier phase may be a liquid, semi-solid (i.e. a gel), or a solid (i.e. a solid implant or scaffold). In one preferred embodiment, the carrier phase is a gel or hydrogel. In one embodiment, the gel or hydrogel is crosslinked. In one embodiment, the hydrogel is a hyaluronan hydrogel. In one embodiment, the carrier phase is a crosslinked hyaluronan hydrogel. In one embodiment, the composition is formulated for direct bladder instillation.

Thus, according to a first aspect of the present invention, there is provided a composition comprising hyaluronan particles dispersed throughout a hyaluronan hydrogel matrix.

In one embodiment, the composition is formulated for direct bladder instillation in a mammal, especially a human. Thus, the hydrogel composition is suitable for being delivered to the bladder using a suitable delivery device such as a catheter and thus is generally a flowable liquid. In other embodiments, the composition may be more solid than liquid, which would make the material suitable for other therapies.

In one embodiment, the hyaluronan particles comprise high molecular weight hyaluronan. In one embodiment, the hyaluronan hydrogel matrix comprises high molecular weight hyaluronan.

In one embodiment, the HA particles are nano-sized particles, typically having an average particle size of 100-900 nm. In one embodiment, the HA particles have an average size of 300 to 700 nm. In one embodiment, the HA particles have an average size of 400 to 600 nm. In one embodiment, the HA particles are agglomerates of nano-sized HA particles, which agglomerates may have an average dimension of 500 nm to 10 microns.

In one embodiment, a weight ratio of HA particles to HA hydrogel matrix is about 1:9 to 9:1. In one embodiment, the weight ratio of HA particles to HA hydrogel matrix is about 1:5 to 5:1. In one embodiment, the weight ratio of HA particles to HA hydrogel matrix is about 1:4 to 4:1. In one embodiment, the weight ratio of HA particles to HA hydrogel matrix is about 1:3 to 3:1. In one embodiment, the weight ratio of HA particles to HA hydrogel matrix is about 1:2 to 2:1. In one embodiment, the weight ratio of HA particles to HA hydrogel matrix is about 1:1. In one embodiment, the HA particles are suspended in the HA gel.

In one embodiment, the HA particles are crosslinked with a crosslinking moiety. In one embodiment, the HA hydrogel matrix is crosslinked with a crosslinking moiety.

In one embodiment, the crosslinking moiety of the HA particles are different to the crosslinking moiety of the HA hydrogel matrix. Use of different crosslinking agents in the particles and hydrogel matrix provides for a composition having a tailored HA degradation profile, and allows the use of different crosslinking agents to provide for a tuneable HA hydrogel scaffold.

In one embodiment, the HA is chemically crosslinked. In one embodiment, the crosslinking moiety (agent) is a functionalised ethylene glycol, for example a functionalised PEG, for example a PEG-amine. In one embodiment, crosslinking initiation is performed with EDC/NHS or 4-(4,6-dimethoxy-1,3,5-triazin-2-yl)-4-methylmorpholinium chloride (DMTMM) chemistry. Other methods of crosslinking include thermal crosslinking or photo-crosslinking. In one embodiment, the ratio of crosslinking agent to HA is 1:1 to 1:10 (by weight), typically 1:1 to 1:5, and preferably about 1:1 to 1:3. In one embodiment, the ratio of crosslinking agent to HA is about 1:2 (by weight).

In one embodiment, the hyaluronan (HA) is positively charged. This can be achieved by derivatizing the HA with a moiety that imparts a net positive charge on the HA molecule (for example a cation). Examples of moieties that can be employed to derivatize HA include aminopropyl imidazole. In this specification, the term HA includes both derivatized and non-derivatized HA. Method of producing positively charged HA, for example cationized HA, are described in the literature and include carboxyl and hydroxyl group modification using quaternary ammonium containing groups (US2009/0281056 and US2010/0197904).

In one embodiment, the composition comprises a therapeutically effective amount of HA. In one embodiment, the composition comprises about 0.1 to about 10% HA (weight %). In one embodiment, the composition comprises about 0.5 to about 5% HA (weight %). In one embodiment, the composition comprises about 0.1 to about 1% HA (weight %). In one embodiment, the composition comprises about 1.0 to about 10% HA (weight %). In one embodiment, the composition comprises about 0.5 to about 2% HA (weight %). In one embodiment, the composition comprises about 5.0 to about 10% HA (weight %).

According to a further aspect of the present invention, there is provided a composition comprising hyaluronan particles disposed within a carrier phase.

The carrier phase is typically a liquid, for example an aqueous fluid. However, the carrier phase may also take the form of a solid or semi-solid phase, for example a gel, hydrogel or polymeric carrier or a matrix formed from a pharmaceutical excipient. The carrier phase may be configured for release of the hyaluronan particles. The carrier phase may be biodegradable, for example water soluble. The carrier phase may be biocompatible. The carrier phase may comprise at least two carrier phases, each phase configured to release the hyaluronan particles are a different release rate. The composition may be an implant for use in the mammalian body. The implant may be solid or semi-solid (for example a scaffold, a gel, a capsule). Polymers suitable for the carrier phase (gel or hydrogel carrier phase) are described in US2016/038643 (especially paragraphs 61 to 63).

In one embodiment, the composition is formulated for direct bladder instillation in a mammal, especially a human. Thus, the composition is suitable for being delivered to the bladder using a suitable delivery device such as a catheter and thus is generally a flowable liquid (i.e. sufficiently flowable for administration through a urinary catheter). In other embodiments, the composition may be more solid than liquid, which would make the material suitable for other therapies.

In one embodiment, the hyaluronan particles comprise high molecular weight hyaluronan.

In one embodiment, the HA particles are nano-sized particles, typically having an average particle size of 100-900 nm. In one embodiment, the HA particles have an average size of 300 to 700 nm. In one embodiment, the HA particles have an average size of 400 to 600 nm. In one embodiment, the HA particles are agglomerates of nano-sized HA particles, which agglomerates may have an average dimension of 500 nm to 10 microns.

In one embodiment, the HA particles are crosslinked with a crosslinking moiety.

In one embodiment, the HA particles are chemically crosslinked. In one embodiment, the crosslinking moiety (agent) is a functionalised ethylene glycol, for example a functionalised PEG, for example PEG-amine. In one embodiment, crosslinking initiation is performed with EDC/NHS or 4-(4,6-dimethoxy-1,3,5-triazin-2-yl)-4-methylmorpholinium chloride (DMTMM) chemistry. Other methods of crosslinking include thermal crosslinking. In one embodiment, the ratio of crosslinking agent to HA is 1:1 to 1:10 (by weight), typically 1:1 to 1:5, and preferably about 1:1 to 1:3. In one embodiment, the ratio of crosslinking agent to HA is about 1:2 (by weight).

In one embodiment, the hyaluronan (HA) is positively charged. This can be achieved by derivatizing the HA with a moiety that imparts a net positive charge on the HA molecule (for example a cation). Examples of moieties that can be employed to derivatize HA include aminopropyl imidazole. In this specification, the term HA includes both derivatized and non-derivatized HA. Method of producing positively charged HA, for example cationized HA, are described in the literature and include carboxyl and hydroxyl group modification using quaternary ammonium containing groups (US2009/0281056 and US2010/0197904).

In one embodiment, the composition comprises a therapeutically effective amount of HA. In one embodiment, the composition comprises about 0.1 to about 10% HA (weight %). In one embodiment, the composition comprises about 0.5 to about 5% HA (weight %). In one embodiment, the composition comprises about 0.1 to about 1% HA (weight %). In one embodiment, the composition comprises about 1.0 to about 10% HA (weight %). In one embodiment, the composition comprises about 0.5 to about 2% HA (weight %). In one embodiment, the composition comprises about 2 to about 4% HA (weight %). In one embodiment, the composition comprises about 5.0 to about 10% HA (weight %).

According to a further aspect of the invention, there is provided hyaluronan particles formed from high molecular weight hyanuronan, and in which the hyaluronan particles are optionally crosslinked. In one embodiment, the hyaluronan particles are nano-sized.

In one embodiment, the nano-sized hyaluronan particles have an average dimension of 300-700 nm. In one embodiment, the nano-sized hyaluronan particles have an average dimension of 400-600 nm.

In one embodiment, the hyaluronan particles are chemically crosslinked, although other methods of crosslinking are possible (for example thermal or photoactivatable crosslinking). In one embodiment, the hyaluronan particles are chemically crosslinked with a functionalised ethylene glycol crosslinking agent, for example a functionalised PEG (i.e. PEG-amine).

In one embodiment, the hyaluronan particles are modified to be positively charged (for example by means of cationisation).

In another aspect, the invention provides a composition of the invention for use as a medicament. In another aspect, the invention provides a composition of the invention for use in a method of treating an inflammatory disease or disorder, typically an inflammatory disease or disorder of an epithelial tissue.

In another aspect the invention provides a composition or particle of the invention for use in a method of treatment of an inflammatory bladder or urinary tract indication in a mammal, wherein the composition or particle is typically administered to the bladder by direct bladder instillation. In another aspect the invention provides a composition or particle of the invention for use in a method of treatment of an indication characterised by GAG layer damage, for example a bladder disease such as interstitial cystitis, painful bladder syndrome, chemical cystitis, radiotherapy-induced cystitis, or recurrent urinary tract infections or feline urinary tract disease (FLUTD).

In one embodiment, the indication is cystitis. In one embodiment, the indication is interstitial cystitis or painful bladder syndrome.

In another aspect the invention provides a composition or particle of the invention for use in a method of GAG replacement therapy.

Other uses of the compositions of the invention include cosmetic, dermatological, tissue regeneration, tissue engineering applications and uses.

In one embodiment, the method of treatment comprises administering the composition or particle periodically during a treatment period. The frequency of administration depends on a number of factors including the status of the disease, the age of the patient, and the effectiveness of the treatment. In one embodiment, the composition or particle is administered once weekly. In one embodiment, the composition or particle is administered once weekly or twice weekly. In one embodiment, the composition or particle is administered once weekly for 4-12 weeks. In one embodiment, the composition or particle is administered daily. In one embodiment, the composition or particle is administered twice monthly. In one embodiment, the composition or particle is administered once monthly. In one embodiment, the composition or particle is administered between 1 and 10 times during the treatment period. In one embodiment, the treatment period is between 1 week and 6 months.

In one embodiment, a unit dose of the composition of the invention (i.e. the amount employed for a single instillation treatment) comprises 10 to 500 mg of (optionally crosslinked) hyaluronan. In one embodiment, a unit dose of the composition of the invention comprises 10 to 200 mg of (optionally crosslinked) hyaluronan. In one embodiment, a unit dose of the composition of the invention comprises 50 to 200 mg of (optionally crosslinked) hyaluronan. In one embodiment, a unit dose of the composition of the invention comprises 100 to 150 mg of (optionally crosslinked) hyaluronan.

The compositions of the invention may include additional components. Thus, the HA particles may comprise one or more additional components. The carrier phase (i.e. hydrogel) may incorporate one or more additional components. Both the HA particles and the carrier phase may, independently, incorporate one or more additional components. The component may be a pharmaceutically or biologically active agent. The component may be a cell, cell component, polysaccharide, protein, peptide, polypeptide, antigen, antibody (monoclonal or polyclonal), antibody fragment (for example an Fc region, a Fab region, a single domain antibody such as a nanobody or VHV fragment), a conjugate of an antibody (or antibody fragment) and a binding partner such as a protein or peptide, a nucleic acid (including genes, gene constructs, DNA sequence, RNA sequence, miRNA, shRNA, siRNA, anti-sense nucleic acid). The component may be a cellular product such as a growth factor (i.e. EGF, HGF, IGF-1, IGF-2, FGF, GDNF, TGF-alpha, TGF-beta, TNF-alpha, VEGF, PDGF and an interleukin. The component may be a drug, for example, a drug to relieve pain such as non-steroidal anti-inflammatory drug (such as Ibuprofen, Ketoprofen or Naproxen), aspirin, acetaminophen, codeine, hydrocodone, an anti-inflammatory agent such as a steroidal anti-inflammatory agent, an anti-depressant, an anti-histamine, or an analgesic. The cell may be autologous, allogenic, xenogenic. The cell may be a stem cell. The stem cell may be selected from the group comprising a side population, embryonic, germinal, endothelial, hematopoietic, myoblast, placental, cord-blood, adipocyte and mesenchymal stem cells. The cells may be engineered to express a biological product, for example a therapeutic biological product such as a growth factor.

In one embodiment, the compositions of the invention have a storage modulus G′ of about 0.1 to 15, preferably about 0.1 to 1. In one embodiment, the compositions of the invention have a loss modulus G″ of about 0.2 to 35, preferably about 0.2 to 1.5. In one embodiment, the compositions of the invention have a complex viscosity (Pa·s) of about 0.1 to 6, preferably about 0.1 to about 0.2.

In another aspect, the invention provides a method of making a composition of the invention comprising the steps of making crosslinked HA particles, making a crosslinked HA hydrogel matrix, and dispersing the crosslinked HA particles in the crosslinked HA hydrogel matrix.

In one embodiment, the HA is crosslinked with a functionalised ethylene glycol crosslinking agent, for example a functionalised PEG crosslinking agent such as PEG-amine. In one embodiment, the method includes a step crosslinking initiation that is typically performed with EDC/NHS or 4-(4,6-dimethoxy-1,3,5-triazin-2-yl)-4-methylmorpholinium chloride (DMTMM) chemistry.

In one embodiment, the ratio of crosslinking agent to HA is 1:1 to 1:10 (by weight), typically 1:1 to 1:5, and preferably about 1:1 to 1:3. In one embodiment, the ratio of crosslinking agent to HA is about 1:2 (by weight).

In one embodiment, the method includes a step of derivatizing the HA with a moiety that imparts a net positive charge on the HA molecule. In one embodiment, the derivatizing step is carried out prior to the crosslinking step.

The invention also relates to a composition obtained by a method of the invention.

Other aspects and preferred embodiments of the invention are defined and described in the other claims set out below.

### BRIEF DESCRIPTION OF THE FIGURES

FIG. 1: Overview of the mode of action of the composition of the invention.

FIG. 2: A comparative analysis between the HA particles and 3 other commercial products (Cystistat, Hyacyst and Ialuril) revealed higher sGAG expression for cells treated with HA particles.

FIG. 3: Positively charged hyaluronan (HA). Positively charged HA was created using by dissolving the HA in water and adding aminopropyl imidazole to native negatively charged HA solution. DSC demonstrates a charge of −15 mV on the HA while in solution.

FIG. 4: The human urothelial cells were inflamed with protamine sulfate for an hour and then treated with three different HA conditions for 24 hour; HA particles in water, HA gel and a combination of HA gel and HA particles in a 1:1 ratio, to mimic the current treatment in human patients. The total HA concentration used were 2 mg, 1 mg and 0.5 mg accordingly. (P<0.05)

FIG. 5: a) A comparative analysis between the HA particles and 3 other commercial products (Cystistat®, Hyacyst® and Ialuril®) revealed higher sGAG expression for cells treated with HA particles; b) Gene expression for hyaluronan synthesis, HAS2 using quantitative PCR also showed significantly increase (P<0.05) in 2 mg/mL, 1.5 mg/mL and 1 mg/mL treated cells compared to non-treated control.

FIG. 6: Ex vivo model of IC was established to optimize the treatment conditions of IC prior to pre-clinical study in rats. Fresh urinary bladder was dissected from rat and cut into 4 smaller pieces. Tissues were stretched up and pinned on agarose gel. The tissues were divided into normal tissues and tissues with protamine sulphate insults. After 2 days, in the protamine sulphate group the tissues were inflamed with 10 mg/ml of protamine sulphate for an hour. For the treatment HA particles were introduced on the bladder tissues and incubate for 2 hours. For the non-treatment group, the protamine sulphate was removed and replaced with DMEM medium without serum. After 2 hours incubation, the HA particles and DMEM medium (no treatment) were removed, replaced with medium with 1% serum and cultured for another 3 days in the incubator with 37 C, 20%02 and 5%002. After 3 days the tissues were fixed in 4% paraformaldehyde, paraffin blocked and stained with H&E.

FIG. 7: The effect of the viscosity of the HA gel on urothelial cells. A) The effect of the viscosity of the HA gel on the production of sGAG. B) The effect of the viscosity of the HA gel on IL-8 levels; C) The effect of the viscosity of the HA gel on IL-6 levels.

FIG. 8: Dynamic oscillatory amplitude, time sweep curves of different HA concentration solutions, storage modulus, G′ (A), Loss modulus, G″ (B), comparison of G′, G″ of different HA concentration solutions (C); SEM image of freeze-dried Hyaluronan solution (3 mg/ml) (D); Rheological viscosity curves (E), comparison of complex viscosity of different HA concentration solutions. Data is represented as Mean±S.D., One way ANOVA, post hoc Tukey test. *p<0.05 vs 1 mg/ml; ****p<0.0001 vs 1 mg/ml

FIG. 9: The effect of 1 mg/ml hyaluronan on inflammatory cytokines. (A) Comparison of different ratios of particles to gel at a HA concentration of 1 mg/ml on secreted IL-6 levels from HTB-2 cells over 24 hours. (B) Comparison of different ratios of different ratios of particles to gel at a HA concentration of 1 mg/ml on secreted IL-8 levels from HTB-2 cells over 24 hours. (C) Comparison of different ratios of different ratios of particles to gel at a HA concentration of 1 mg/ml on secreted MCP-1 levels from HTB-2 cells over 24 hours.

FIG. 10: The effect of 3 mg/ml hyaluronan on inflammatory cytokines. (A) Comparison of different ratios of particles to gel at a HA concentration of 3 mg/ml on secreted IL-6 levels from HTB-2 cells over 24 hours. (B) Comparison of different ratios of ratios of particles to gel at a HA concentration of 3 mg/ml on secreted IL-8 levels from HTB-2 cells over 24 hours. (C) Comparison of different ratios of particles to gel at a HA concentration of 1 mg/ml on secreted MCP-1 levels from HTB-2 cells over 24 hours.

FIG. 11: The effect of the HA in HA system on bladder permeability. (A) Comparison of the effect of different ratios of particles to gel at a HA concentration of 1 mg/ml and Cystistat® on TEER levels over 6 hours on T84 cells treated with protamine sulphate. (B) Comparison of the effect of different ratios of particles to gel at a HA concentration of 1 mg/ml and Cystistat® on TEER levels over 6 hours on T84 cells treated with protamine sulphate and TNFα. (C) Comparison of the effect of different ratios of particles to gel at a HA concentration of 3 mg/ml on Papp over 6 hours on T84 cells treated with protamine sulphate (D) Comparison of the effect of different ratios of particles to gel at a HA concentration of 3 mg/ml on Papp over 6 hours on T84 cells treated with protamine sulphate and TNFα. (E) Comparison of the effect of different ratios of particles to gel at a HA concentration of 3 mg/ml and Cystistat® on TEER levels over 6 hours on T84 cells treated with protamine sulphate. (F) Comparison of the effect of different ratios of particles to gel at a HA concentration of 3 mg/ml different 3 mg/ml and Cystistat® on TEER levels over 6 hours on T84 cells treated with protamine sulphate and TNFα.

FIG. 12: FIG. 1: NMR-H for cross linked particles using as coupling reagent: (a) EDC/NHS, (b) DMTMM (4-(4,6-Dimethoxy-1,3,5-triazin-2-yl)-4-methylmorpholine)

FIG. 13: NMR-H of final products obtained under three different reaction conditions after centrifugation at 1500 rpm. Purple: control reaction, coupling reagent was not used; Red: EDC was used as coupling reagent; Green: DMTMM was used as coupling reagent.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction of inflammation of human urothelial cells). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human.

As used herein, the term “hyaluronan” or “hyaluronic acid” or “HA” refers to the anionic non-sulphated glycosaminoglycan that forms part of the extracellular matrix in humans and consists of a repeating disaccharide→4)-β-d-GlcpA-(1→3)-β-d-GlcpNAc-(1→. Hyaluronan is the conjugate base of hyaluronic acid, however the two terms are used interchangeably. When a salt of hyaluronic acid is employed, the sale is generally a sodium salt, although the salt may be employed such a calcium or potassium salts. The hyaluronic acid or hyaluronan may be obtained from any source, including bacterial sources. Hyaluronic acid sodium salt from Streptococcus equi is sold by Sigma-Aldrich under the product reference 53747-1G and 53747-10G. Microbial production of hyaluronic acid is described in Liu et al (Microb Cell Fact. 2011; 10:99). The term also includes derivatives of HA, for example HA derivatised with cationic groups as disclosed in US2009/0281056 and US2010/0197904, and other types of functionalised derivatives, such as the derivatives disclosed in Menaa et al (J. Biotechnol Biomaterial S3:001 (2011)), Schante et al (Carbohydrate Polymers 85 (2011)), EP0138572, EP0216453, EP1095064, EP0702699, EP0341745, EP1313772 and EP1339753.

As used herein, the term “hyaluronan hydrogel matrix” means a three dimensional network of hyaluronan polymers in a water dispersion medium. In one embodiment, the hyaluronan polymers are crosslinked to form the three-dimensional network. In one embodiment, the matrix is formed with a homopolymer, typically a HA homopolymer. In none embodiment, the matrix is a single gel system (as opposed to the two-gel system of EP2011816).

As used herein, the term “high molecular weight” as applied to hyaluronic acid means a molecular weight of greater than 500 KDa. In one embodiment, the high molecular weight has a molecular weight of greater than 600 KDa. In one embodiment, the high molecular weight has a molecular weight of greater than 700 KDa. In one embodiment, the high molecular weight has a molecular weight of greater than 800 KDa. In one embodiment, the high molecular weight has a molecular weight of greater than 900 KDa. In one embodiment, the high molecular weight has a molecular weight of greater than 1000 KDa. In one embodiment, the high molecular weight has a molecular weight of greater than 1100 KDa. In one embodiment, the high molecular weight hyaluronan has a molecular weight of between 500 and 5000 KDa. In one embodiment, the high molecular weight hyaluronan has a molecular weight of between 500 and 2000 KDa. In one embodiment, the high molecular weight hyaluronan has a molecular weight of between 500 and 1500 KDa. In one embodiment, the high molecular weight hyaluronan has a molecular weight of between 500 and 1000 KDa

As used herein, the term “crosslinked” as applied to hyaluronic acid means that hyaluronic acid polymer chains are covalently crosslinked with a crosslinking agent (moiety) to form a three-dimensional network. Crosslinked HA hydrogels are described in the literature, for example in Kenne et al (Carbohydrate Polymers, Vol. 91, Issue 1 (2011)), Segura et al (Biomaterials, Vol. 26, Issue 4 (2005)), Yeom et al (Bioconjugate Chem, Vol. 21(2) 2010), U.S. Pat. Nos. 8,124,120, and 6,013,679. The term “crosslinking agent” means a molecule containing two or more functional groups that can react with HA. Examples of crosslinking agents include functionalised ethylene glycol crosslinking agents, including functionalised polyethylene glycol (PEG), for example PEG-amine and PEG diglycidylether (EX810), 1-ethyl-3-(3-dimethylaminopropyl) carbimide (EDC), divinyl sulfone (DVS) and ethylene glycol diacrylates and dimethacrylates, derivatives of methylenebisacrylamide (Sigma-Aldrich). Other examples of crosslinking agents are described in WO2009//018076. In one embodiment, the HA, for example the HA hydrogel, is crosslinked in situ in the body. The hydrogel and crosslinking agent may be kept separate prior to administration, and combined during or after administration to form crosslinked HA. A duploject injection system may be employed to crosslink the hydrogel in-situ.

As used herein, the term “nano-sized” as applied to hyaluronan particles means having an average dimension in the nanometer range. For example, the HA particles may have an average size of 1 to 1000 nm, typically 100 to 900 nm, typically 200 to 800 nm, preferably 300 to 700 nm, and more preferably 400 to 600 nm. In one embodiment, the HA particles have an average size of 500+/−100 nm. Particle size is measured using a Malvern Zetasizer (nano range).

As used herein, the term “dispersed and suspended” as applied to the HA particles in the carrier phase (i.e. crosslinked HA hydrogel) means that the particles are encapsulated within the gel as opposed to being co-crosslinked with the gel as is described in US2010/028435.

As used herein, the term “formulated for direct bladder instillation” means that the composition is sufficiently fluid to allow it to be instilled into a human bladder through a bladder instillation device, for example a catheter. Bladder instillation compositions will be well known to the person skilled in the art, and it would be a routine matter for a person skilled in the art to formulate a composition of the invention for bladder instillation. Methods of performing bladder instillation is well known to a person skilled in the art and is described in the following documents: U.S. Pat. Nos. 5,880,108, 5,888,986, 5,994,357, 26,635,625. In one embodiment, the composition of the invention is formulated for bladder instillation using a 8F or 10F catheter.

As used herein the term “pharmaceutically or biologically active agent” refers generally to an agent or component that has a pharmaceutical or biological effect in a mammal. Examples include cells, cell components, polysaccharides, proteins, peptides, polypeptides, antigen, antibody (monoclonal or polyclonal), antibody fragment s (for example an Fc region, a Fab region, a single domain antibody such as a nanobody or VHV fragment), a conjugate of an antibody (or antibody fragment) and a binding partner such as a protein or peptide, a nucleic acid (including genes, gene constructs, DNA sequence, RNA sequence, miRNA, shRNA, siRNA, anti-sense nucleic acid), cellular products such as growth factors (i.e. EGF, HGF, IGF-1, IGF-2, FGF, GDNF, TGF-alpha, TGF-beta, TNF-alpha, VEGF, PDGF and an interleukin), drugs, for example, a drug to relieve pain such as non-steroidal anti-inflammatory drug (such as Ibuprofen, Ketoprofen or Naproxen), aspirin, acetaminophen, codeine, hydrocodone, an anti-inflammatory agent such as a steroidal anti-inflammatory agent, an anti-depressant, an anti-histamine, or an analgesic. The cell may be autologous, allogenic, xenogenic. The cell may be a stem cell. The stem cell may be selected from the group comprising a side population, embryonic, germinal, endothelial, hematopoietic, myoblast, placental, cord-blood, adipocyte and mesenchymal stem cells. The cells may be engineered to express a biological product, for example a therapeutic biological product such as a growth factor.

As used herein, the term “Inflammatory disorder” or “inflammatory disease” means an immune-mediated inflammatory condition that affects mammals especially humans and is generally characterised by dysregulated expression of one or more cytokines. Examples of inflammatory disorders include skin inflammatory disorders, inflammatory disorders of the joints, inflammatory disorders of the vertebrae and/or vertebral discs, inflammatory disorders of the cardiovascular system, certain autoimmune diseases, lung and airway inflammatory disorders, intestinal inflammatory disorders. Examples of skin inflammatory disorders include dermatitis, for example atopic dermatitis and contact dermatitis, acne vulgaris, and psoriasis. Examples of inflammatory disorders of the joints include rheumatoid arthritis. Examples of inflammatory disorders of the intervertebral discs include intervertebral disc degeneration. Examples of inflammatory disorders of the cardiovascular system are cardiovascular disease, atherosclerosis and critical limb ischemia. Examples of autoimmune diseases include Type 1 diabetes, Graves disease, Guillain-Barre disease, Lupus, Psoriatic arthritis, Ulcerative colitis and crohn's disease. Examples of lung and airway inflammatory disorders include asthma, cystic fibrosis, COPD, emphysema, and acute respiratory distress syndrome. Examples of intestinal inflammatory disorders include colitis and inflammatory bowel disease. Other inflammatory disorders include cancer, hay fever, periodontitis, allergies, hypersensitivity, ischemia, depression, systemic diseases, post infection inflammation and bronchitis. In this specification, the term “Metabolic disorder” should be understood to include pre-diabetes, diabetes; Type-1 diabetes; Type-2 diabetes; metabolic syndrome; obesity; diabetic dyslipidemia; hyperlipidemia; hypertension; hypertriglyceridemia; hyperfattyacidemia; hypercholerterolemia; hyperinsulinemia, and MODY.

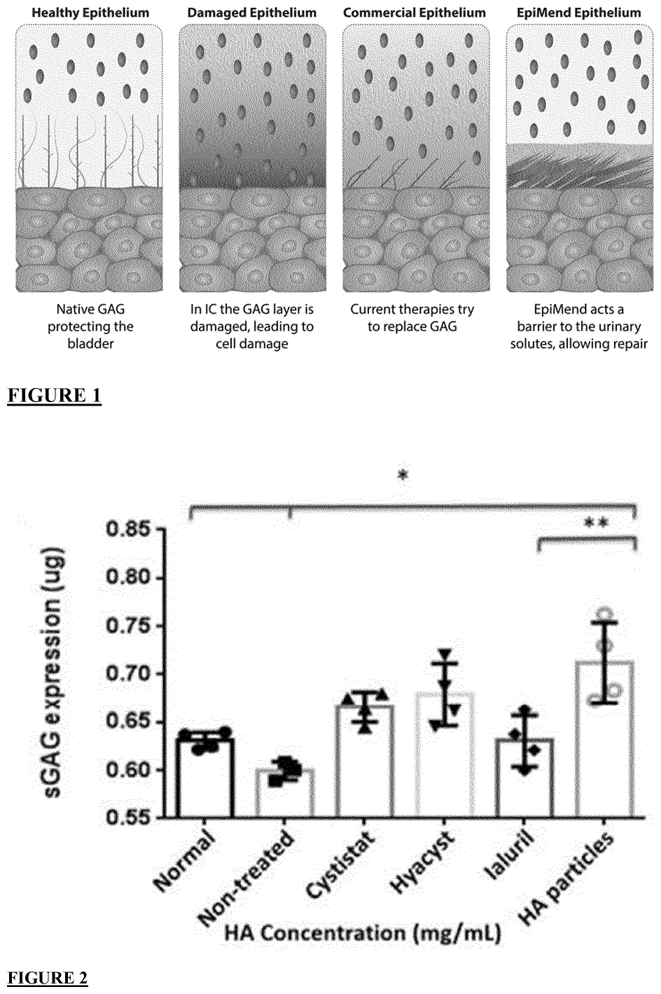

Interstitial Cystitis (IC) is a chronic disease that is characterised by disruption of the bladder native GAG barrier, glycosaminoglycan (GAG) barrier layer, increasing permeability to noxious components from urine which is currently refractory to effective treatment. One composition of the invention comprises of hyaluronan-based particles in a hyaluronan-based hydrogel formulated for instillation into the bladder to repair the damaged GAG layer for the treatment of epithelial barrier disorders, such as IC. The composition of the invention provides a more effective treatment for IC and will be used by clinicians in the same way as currently marketed therapies, as a device delivered by non-surgical catheterisation. The hyaluronan hydrogel will act as a GAG rich delivery system, also binding the cell wall and forming a barrier effect to facilitate repair of the GAG layer. The HA particles will increase the distance between the urinary solutes and the bladder wall. The tailored degradation profile of the composition of the invention will increase bioavailability in the bladder, increase barrier function and residence time thus facilitating restoration/regeneration of the luminal lining and suppression of inflammatory cytokine production thus acting as a first line therapy for IC which addresses the underlying disease pathology of IC. An overview of the mode of action of the composition of the invention in the treatment of interstitial cystitis is provided in FIG. 1.

GAG replacement instillation therapies aim to repair the wall by replacing the native GAG layer that is lost. Nonetheless the optimal regimen has not yet been defined—with varying multiple weekly doses for initial response, along with maintenance treatment—requiring repeated hospital visits. Thus the treatment of the invention comprising of a hyaluronan gel containing hyaluronan particles will demonstrate a significant efficacy profile relative to existing intravesical hyaluronan solution instillation therapies. The treatment of the invention will bind in the same manner as the current high molecular weight HA treatment, however gradual degradation of the HA particles increases residence time, barrier function and bioavailability of HA in bladder, while also stimulating endogenous GAG production. The composition of the invention increases separation distance between bladder and urinary solutes, decreasing the effect of urinary solutes on the activated bladder wall and decreasing cytokine secretion. Additionally the positively charged HA, will have a stronger affinity for the negatively charged urothelium, thus increasing residence time and barrier functions. Thus the treatment of the invention will exhibit an enhanced clinical efficacy profile relative to existing therapeutic interventions for IC as shown below in Table 1

TABLE 1
Competitive advantages of the Compositions of the Invention

| | | Tribute | Institut | | | |
|---|---|---|---|---|---|---|
| | Pohl- | Pharma | Biochimique | Syner-Med | | |
| Company | Boskamp | Canada Inc | SA | Pharma | Mylan | Invention |
| Product | Gepan ® | Uracyst ® | iAluRil ® | Hyacyst ® | Cystistat ® | EpiMend |
| Name | | | | | | |
| Active | Chondroitin | Sodium | LMW-HA and | LMW-HA | HMW-HA | HMW-HA |
| Component | Sulphate | Chondroitin | Chondroitin | | | |
| | | Sulfate | Sulphate | | | |
| Composition | Fluid | Fluid | Fluid | Fluid | Fluid | HMW-HA |
| | | | | | | particles |
| | | | | | | embedded in |
| | | | | | | HMW-HA gel |
| Clinical trial | Double Blinded | Double Blinded | Double Blinded | Temporary | Increases cell | Increases cell |
| | Placebo Control | Placebo Control | Placebo Control | LMW-HA has | endogenous | endogenous |
| | studies show | studies show | studies show | no effect on | production | production of |
| | lack of clinical | lack of clinical | lack of clinical | GAG | of GAGs | GAGs leading |
| | effect | effect | effect | production | leading to | to repair |
| | | | | | repair | |
| Evidence | Increases | Increases | LMW-HA | LMW-HA | HMW-HA | HMW-HA |
| | permeability of | permeability of | induces pro- | induces pro- | reduces pro- | reduces pro- |
| | cells to | cells to | inflammatory | inflammatory | inflammatory | inflammatory |
| | inflammatory | inflammatory | secretion | Secretion | secretion | secretion |
| | agents [21] | agents | | | | |
| Typical Dose | Weekly | Weekly | Weekly | Biweekly | Biweekly | Weekly or |
| Regime | | | | | | Biweekly |
| Route of | Non-surgical | Non-surgical | Non-surgical | Non-surgical | Non-surgical | Non-surgical |
| application | catheterisation | catheterisation | catheterisation | catheterisation | catheterisation | catheterisation |
| Potential for | No | No | No | No | No | Yes- |
| controlled | | | | | | Therapeutics |
| therapeutic | | | | | | can be |
| release | | | | | | encapsulated in |
| | | | | | | HA particles to |
| | | | | | | complement |
| | | | | | | GAG |
| | | | | | | replacement |

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1: Protocol for Non-Cross Linked, Crosslinked HA Hydrogels

### Material Specifications:

Hyaluronic acid: High molecular weight (HM Wt.) sodium hyaluronate 1 M. Da (Lifecore Biomedical, USA). CAS No.: 9067-32-7.

PEG-amine: Mw 2000 Da purchased from JenKem Technology USA (Allen, Tex.). CAS No.: 25322-68-3, purity >95%.

N-(3-Dimethylaminopropyl)-N′-ethylcarbodiimide hydrochloride (EDC) (Sigma-Aldrich, USA) CAS Number 25952-53-8, purity ˜100%.

N-hydroxysuccinimide (NHS): (Sigma-Aldrich USA). CAS Number 6066-82-6, purity 98%.

Phosphate buffered saline (Sigma-Aldrich, USA) CAS Number P4417-50TAB (pH adjusted to 6.5)

### Formulation or Compounding Procedure for Non-Cross Linked Gels:

1. Prepare Phosphate buffered saline by dissolving 1 tablet in 200 ml distilled water, and adjust the pH to 6.5 (store aside).

2. Slowly add required quantities of Hyaluronic acid sodium salt (1 mg/ml, 3 mg/ml, 9 mg/ml, 15 mg/ml) in Phosphate buffered saline at 25° C.

3. Stir the solutions over-night on a magnetic stirrer at 25° C. Check the pH if it is within the limit 6.5-7.5.

4. Store the hydrogels at cold room conditions (4° C.) for further use.

### Formulation or Compounding Procedure for Cross Linked Gels:

1. Slowly add Hyaluronic acid sodium salt at 9 mg/ml in 0.1 M MES buffer (pH 6.0).

2. To the above solution add EDC (4.5 mg) and NHS (2.7 mg) (with respect to each monomer of HA repeating units) in MES buffer and stirred for 15 minutes. Continue mixing, add required quantity of PEG amine (1:1.32 ratio with respect to HA) for cross-linking and the reaction was stirred overnight at 25° C.

3. After completion, the reaction mixture was dialyzed for 24-48 h against distilled water using 6000-8000 MW dialysis membrane to remove any unreacted starting materials and salts.

After dialysis the samples were freeze dried and lyophilized to obtain pure cross-linked HA hydrogels. Redisperse the lyophilized powder in PBS to get same concentration of the gels

### Example 2: Protocol for Crosslinking of HA

### Material Specifications

Hyaluronic acid: High molecular weight (HMwt) sodium hyaluronate 1.2×10⁶ Da (Lifecore Biomedical, USA). CAS No.: 9067-32-7.

PEG-amine: Mw 2000 Da purchased from JenKem Technology USA (Allen, Tex.). CAS No.: 25322-68-3, purity >95%

N-(3-Dimethylaminopropyl)-N′-ethylcarbodiimide hydrochloride (EDC) (Sigma-Aldrich USA) CAS Number 25952-53-8, purity ˜100%

N-hydroxysuccinimide (NHS): (Sigma-Aldrich USA). CAS Number 6066-82-6, purity 98% Solvents: 20 wt % sodium sulphate solution in distilled water and 0.1 M MES (2-(N-morpholino)ethanesulfonic acid) buffer

### Evaluation of Synthetic Protocol for Cross-Linking of HA (EDC/NHS)

1. 10 mg/mL conc. HA was dissolved in 0.1 M MES buffer for 2 h at room temperature

a. MES buffer facilitates rapid dissolution of HA to obtain a homogeneous solution

b. MES buffer (pH-6) also facilitates ionization of the carboxylic groups of HA (pKa-3-4)

2. A solution of 20 wt % Na2SO4, a neutral ionic salt was then added to further induce ionisation of HA

3. To this solution were added solutions of EDC (46 mg, 0.2 M) and NHS (27 mg, 0.2 M) in MES buffer and stirred for 15 minutes

a. MES buffer has been reported in literature to increase the cross-linking efficiency and the yield

4. PEG (5 mg, 1:2 ratio wrt. HA) was then added for cross-linking and the reaction was stirred overnight at RT

5. After completion the reaction mixture was dialyzed for 48 h against distilled water using a 6000-8000 MW dialysis membrane to remove any unreacted starting materials and salts for 48 h

6. After dialysis the samples were freeze dried and lyophilized to obtain pure cross-linked HA particles

7. Characterization by Zeta Potential and SEM

a. An increase in the size and a decrease in the zeta potential was observed and is attributed to the increased cross-linking efficiency as this reduces the number of free carboxylic groups and also the length of the particles

8. Zeta Potential: −24.8 (Mean), 2.17 (Std Dev), −21.6 (max), −27.3 (min)

### Evaluation of Synthetic Protocol for Cross-Linking of HA (DMTMM)

1. 10 mg/mL conc. HA was dissolved in 0.1 M MES buffer for 2 h at room temperature

a. MES buffer facilitates rapid dissolution of HA to obtain a homogeneous solution

b. MES buffer (pH-6) also facilitates ionization of the carboxylic groups of HA (pKa-3-4)

2. A solution of 20 wt % Na2SO4, a neutral ionic salt was then added to further induce ionisation of HA

3. To this solution add a solution of DMTMM (7.3 mg, 1 eq. wrt HA repeating unit)

a. MES buffer has been reported in literature to increase the cross-linking efficiency and the yield

4. PEG (5 mg, 1:2 ratio wrt. HA) was then added for cross-linking and the reaction was stirred overnight at RT

5. DMTMM and Peg-amine were dissolved in 0.1 M MES Buffer to make a total volume of 1 mL, final reaction volume was 3 mL

6. The reaction was enriched with 8 vol % saturated sodium chloride (5 mL)

7. The product was precipitated using 96% ethanol (10 mL)

8. The reaction was allowed to stir for 30 mins to allow complete precipitation

9. Centrifuged at 1500 rpm for 5 mins to collect product

10. Several wash steps were subsequently performed and then the product was filtered and kept under vacuum for 48 h

11. The samples were characterization by NMR for purity, TNBSA for degree of cross-linking and SEM for morphological evaluation.

The crosslinked HA particles are mixed with the HA gel to encapsulate the HA particles in the HA gel or hydrogel for further testing

### Example 3

Human urothelial cells HTB4 were grown in basal media consisting of Dulbecco's Modified Eagle's Medium (DMEM) supplemented with 10% fetal calf serum (FCS) and 1% penicillin/streptomycin. In all cases cells were grown until 90-100% confluent and washed three times by rinsing with Phosphate Buffer Solution (PBS) before treatment. Monolayer cells were chemically stripped using protamine sulfate (100 ng/ml) for 30 minutes to remove the GAG layer. After stripping, the cells were washed by rinsing three times with PBS and then allocated to treatment groups. The treated groups were basal media (control), HA particles (1 mg/ml), three commercial products from Cystistat, Hyacyst and Ialuril and no treatment control (normal GAG layer or no pre-treatment with protamine sulfate). The cells were treated for 1 hour and then rinsed with PBS and replaced with DMEM medium without serum and incubate for another 24 hour. After 24 hour the supernatant were collected and subjected to Blyscan sulfated glycosaminoglycan assay (Biocolor.co.uk) to measure the secreted sGAG production.

A comparative analysis of sGAG expression between the HA particles and three other commercial products (Cystistat, Hyacyst and Ialuril) revealed a significant increase (P<0.05) in sGAG expression for the HA particles compared to normal cells and non-treated cells (FIG. 2).

### Example 4

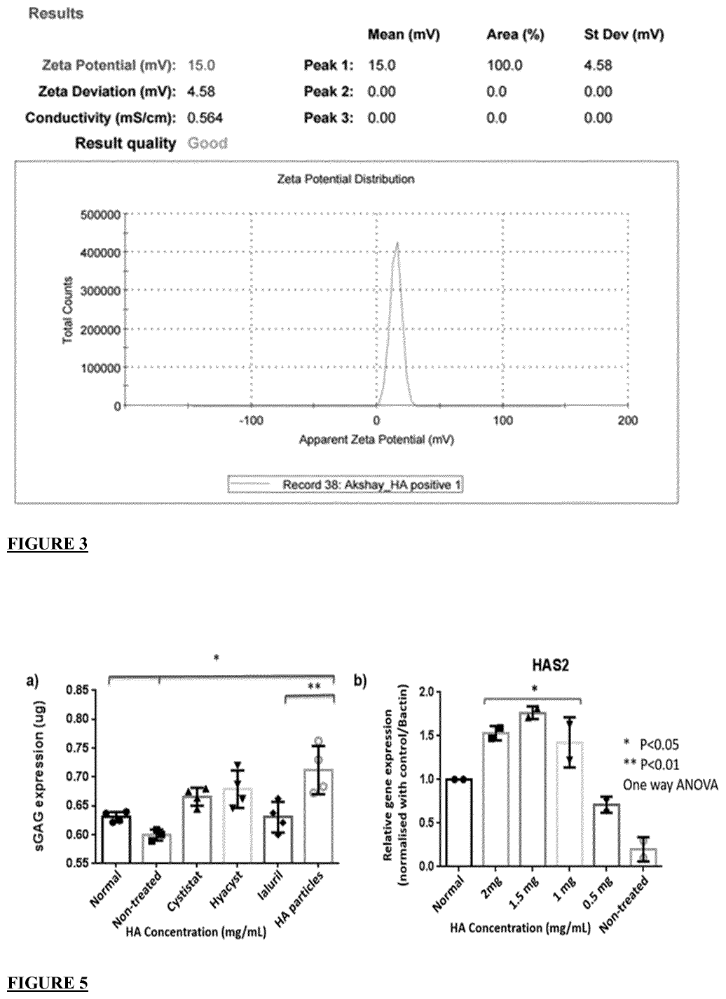

Positively charged HA was created using by dissolving the HA in water and adding aminopropyl imidazole to native negatively charged HA solution followed by the addition of EDC/NHS or 4-(4,6-dimethoxy-1,3,5-triazin-2-yl)-4-methylmorpholinium chloride (DMTMM) to the solution followed by incubation of the solution for 24-48 h under constant stirring. The polymer was purified by dialysing it against water and lyophilized to obtain the powder form. (FIG. 3)

### Example 5

The efficacy of HA particles was tested for its ability to increases sGAG compared with HA in a gel format and compared to HA gel containing particles. HTB4 cells were cultured as stated above. The cells were chemically stripped using protamine sulfate (100 ng/ml) for 30 minutes to remove the GAG layer. After stripping, the cells were rinsed three times with PBS and then allocated to treatment groups. The treated groups were basal media (control), 3 different HA groups; HA particles only, HA gel only and combination (HA particles and HA gel) with the concentration of 0.5, 1 and 2 mg/ml respectively and no treatment control (GAG layer was not removed or no pre-treatment with protamine sulfate). The cells were treated for 1 hour and then the cells were rinsed with PBS and replaced with DMEM medium without serum and incubate for 24 hour. After 24 hour the supernatant were collected and subjected to Blyscan sulfated glycosaminoglycan assay (Biocolor.co.uk) to measure the secreted sGAG production.

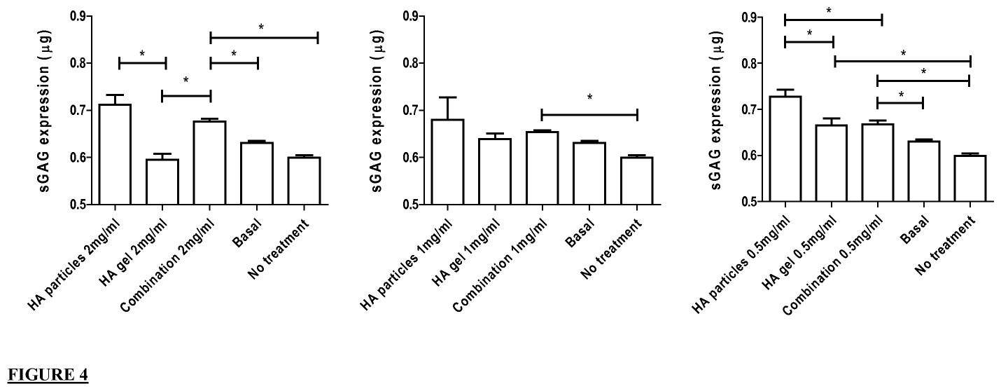

FIG. 4 demonstrate that the particles increase the cells production of sGAG. The particles alone having significant effect compared to cells with no treatment. However all of the concentrations of HA particles and gel have a dramatic and consistent effect on increases sGAG levels.

### Example 6

The ability of HA particles to increase sGAG levels compared to commercially available treatments was examined. Treated and control HTB4 cells were trypsinised, lysed and RNA was isolated using the RNeasy mini kit (SA Bioscience). mRNA (1500 ng) was reverse transcribed to cDNA using the high capacity cDNA reverse transcription kit (Applied Biosystems). cDNA (75 ng) was added to each RT-PCR reaction in a master mix reagent, FastStart Universal SYBR Green Master (Rox) (Roche). The real time cyclers were performed using StepOne plus system (Applied Biosystems). HAS2 gene expression was calculated as fold change in the comparative CT (ΔΔCT) experiment normalised to the endogenous control, B actin. Analyses were done using the StepOne (Applied Biosystems) software. FIG. 5A shows that HA particles dramatically improve sGAG levels compared to commercial treatments.

FIG. 5B demonstrates using qPCR that a range of concentrations increases the production of HAS2 gene, an enzyme critical for the production of long chain HA. Both graphs in FIG. 5 taken together demonstrate that the particles are increasing the bioavailability of HA and sGAG in the urothelial cells.

### Example 7

3 adults female Sprague Dawley rats weighing from 280-300 g were euthanized and the urinary bladder were harvested. The bladder tissue were then cut into half horizontally, rinsed in phosphate buffer saline (PBS) three times, stretched up and pinned on the 4 corners on a solid agarose basolateral side down and urothelium exposed to the media. The explant were then exposed to protamine sulphate 10 mg/ml for 1 hour, then washed with PBS and then covered in cell culture media or HA particles and culture for another 3 days.

After 3 days, the harvested bladders were then fixed in 4% paraformaldehyde overnight. The tissues were then paraffin embed, sectioned (5 μm) and stained with hematoxylin and eosin. Tissue explants were removed and stabilised on agarose basolateral side down and urothelium exposed to the media.

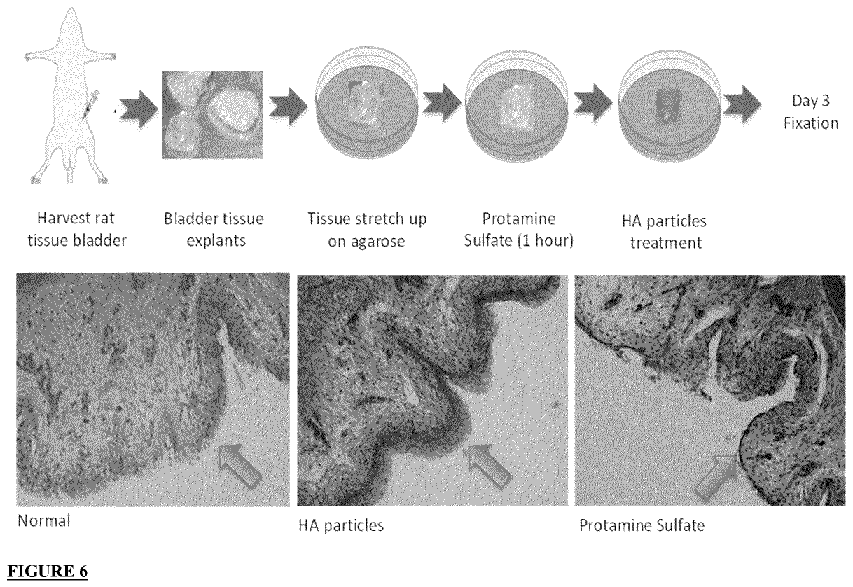

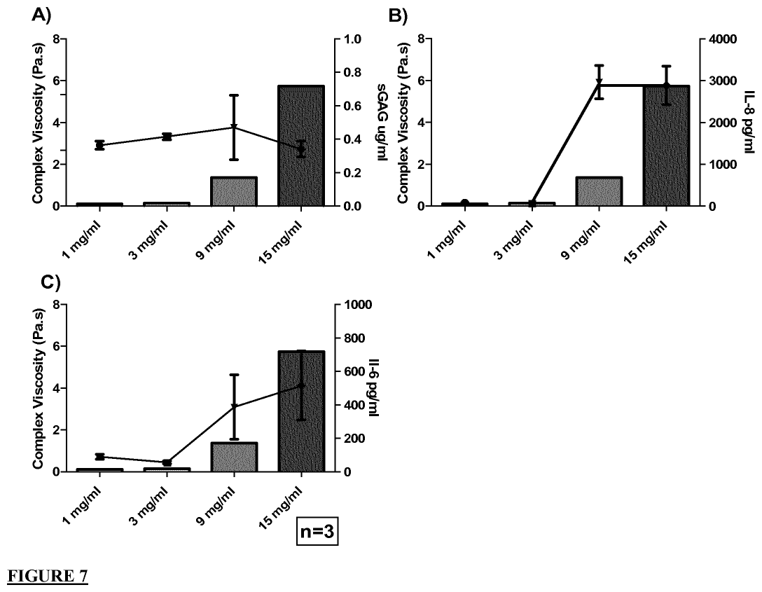

In FIG. 6 shows tissue that has not be exposed to protamine sulphate (Normal), tissue that has been exposed to HA particles (HA particles) and tissue that has been exposed to protamine sulphate for one hour (Protamine sulphate). This shows how the protamine sulphate has degraded the urothelium tissue (blue arrow). The tissue treated with HA particles has retained greater tissue integrity with thicker urothelium compared to both normal and protamine sulphate tissue (blue arrow).

### Example 8

To elucidate the role of the viscosity of HA, we investigated whether a change in viscosity would correlate with a change in a biological effect on the urothelial cells. We studied the effect of 1 mg/ml, 3 mg/ml, 9 mg/ml and 15 mg/ml HA gels on sGAG and IL-6 and IL-8 expression on urothelial cells under normal conditions. We examined the effect of the viscosity of the HA gel on sGAG secretion (FIG. 7A). We observed that an increase in the viscosity of the HA gel did not result in an increase in sGAG secretion. We examined the effect of the effect of the viscosity of the HA gel on IL-8 secretion (FIG. 7B). We observed that increasing the viscosity of the HA gel led to an increase in IL-8 secretion. We examined the effect of the viscosity of the HA gel on IL-6 secretion (FIG. 7C). It was observed that increasing the viscosity of the HA gel led to an increase in IL-6 secretion.

### Example 9

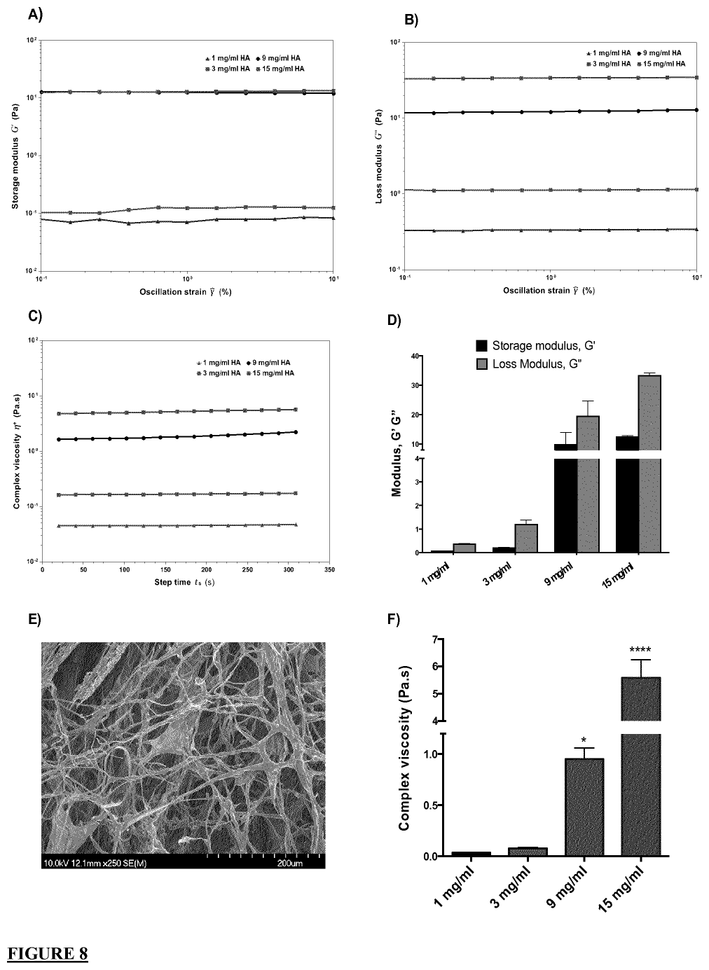

Rheological measurements of hyaluronan solutions were performed with a Discovery Series Rheometer, using parallel plate geometry of 60 mm diameter, the selected geometry was chosen to provide a balance between sensitivity and sample volume. Hyaluronan solutions (1, 3, 9, 15 mg/ml) were vortexed and then each sample directly loaded on the bottom plate, and the upper plate was then lowered to a measurement gap of 500 μm. The measurement parameters were determined to be within the linear viscoelastic region in preliminary experiments by amplitude and frequency sweeps. (FIG. 8). The measurement was allowed to proceed until the storage modulus (G′), loss modulus (G″) reached a plateau. The modulus (G′, G″) and complex viscosity (q*, Pa·s) were taken at 37° C. in the dynamic oscillatory mode with amplitude sweep (0.1 to 10% strain at 1 hz frequency) and time sweep (at 1 Pa stress, 0.1 Hz frequency for 5 minutes).

### Example 10

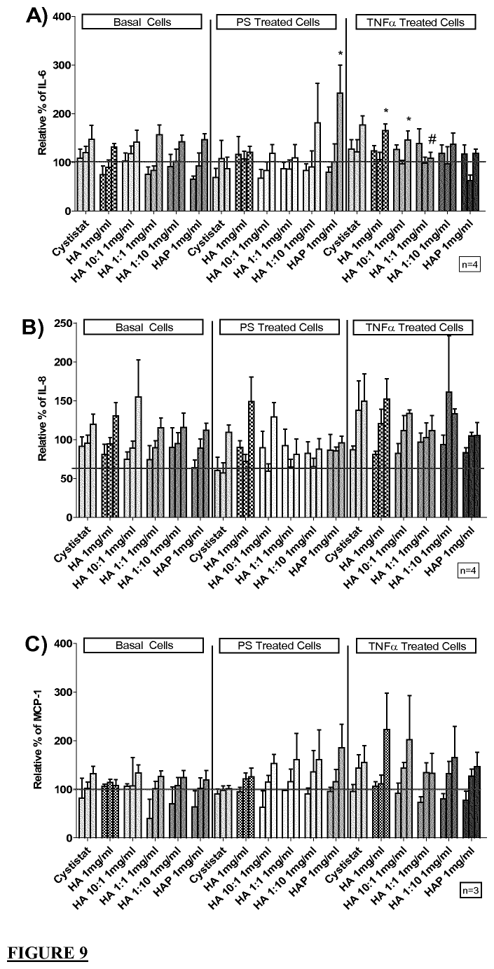

Evaluation of the concentration of the hyaluronan particles in a hyaluronan gel on MCP-1, IL-6 and IL-8 (FIG. 9). Human urothelial cells HTB2 were grown on plastic in basal media consisting of Dulbecco's Modified Eagle's Medium (DMEM) supplemented with 10% foetal calf serum (FCS) and 1% penicillin/streptomycin. Cells were grown until 80-90% confluent and washed three times by rinsing with Hanks' Balanced Salt Solution (HBSS) before all experiments. HTB-2 cells were washed using HBSS and Trypsin-EDTA solution, 0.25% added for 10 minutes, centrifuged for 10 minutes at 1000 rpm. Cells were then divided and seeded into 48 well plates at 50,000 cells per well. Cells were grown for 24 hours in basal media. Experiments were conducted in different conditions. H₂O₂ treated cells: Cell monolayers in 48 well plates were chemically stripped and inflamed using hydrogen peroxide (1% H2O2 in basal media) for one hour before application of the hyaluronan intervention. Protamine sulphate (PS) treated cell monolayers in 48 well plates were chemically stripped using protamine sulphate (100 ng/ml) for one hour before HA intervention. TNFα treated cell monolayers in 48 well plates were inflamed using TNFα (10 ng/ml) for one hour before HA intervention. Basal conditions: Cell monolayers in 48 well plates media was replaced with basal media. HA intervention: Cells were washed with HBSS. Cystistat® (0.8 mg/ml) or control (Basal media) or HA solutions (1 mg/ml, 3 mg/ml, 9 mg/ml, 15 mg/ml) are added to wells for two hours, then washed with HBSS and replaced with basal media for 24 hours. Cell supernatants were removed and stored at −20° C.

### Example 11

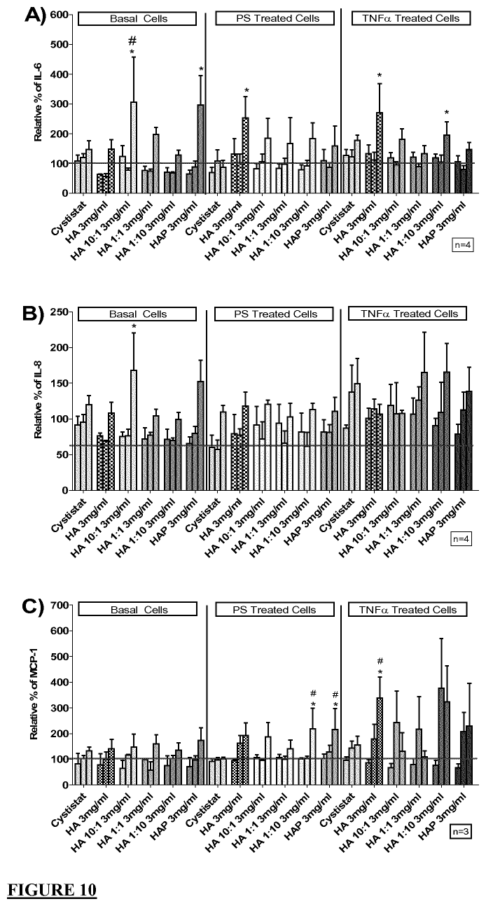

The effect of 3 mg/ml hyaluronan on inflammatory cytokines (FIG. 10) (A) Comparison of different ratios of particles to gel at a HA concentration of 3 mg/ml on secreted IL-6 levels from HTB-2 cells over 24 hours. (B) Comparison of different ratios of ratios of particles to gel at a HA concentration of 3 mg/ml on secreted IL-8 levels from HTB-2 cells over 24 hours. (C) Comparison of different ratios of particles to gel at a HA concentration of 1 mg/ml on secreted MCP-1 levels from HTB-2 cells over 24 hours.

### Example 12

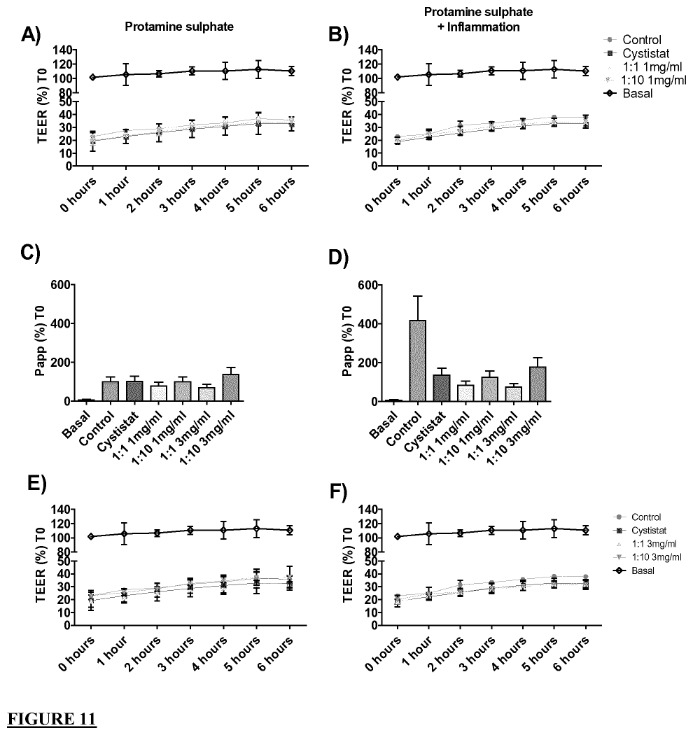

Permeability testing: Transepithelial electrical resistance (TEER) and FITC-Dextran (4 kDa) fluxes across T84 monolayers were measured over 6 hours and the apparent permeability coefficient (Papp) of FITC-Dextran (4 kDa) was calculated. These studies validate the efficacy of the HA particles in a HA gel to provide a barrier effect and decrease the permeability of the bladder wall. (FIG. 11)

### Example 13

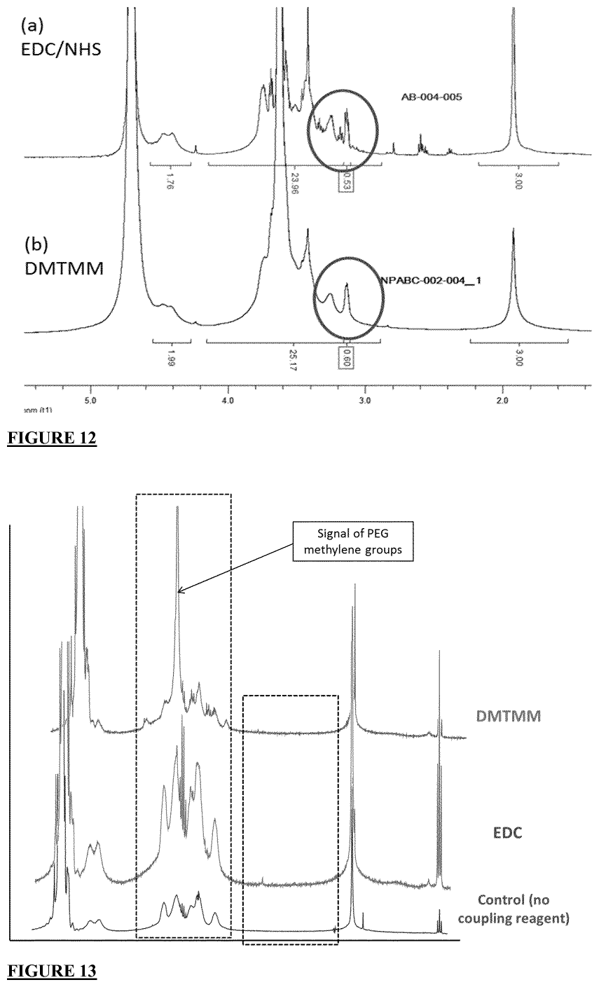

It was found that the degree of cross-linking can be enhanced by using (4-(4,6-Dimethoxy-1,3,5-triazin-2-yl)-4-methylmorpholine) (DMTMM) instead of N-(3-Dimethylaminopropyl)-N′-ethylcarbodiimide; NHS: N-Hydroxysuccinimide (EDC/NHS) as it is not sensitive to change in pH of the solution (FIG. 12). Broader signal at 3.15 ppm when using DMTMM (b) suggests a higher degree of cross-linking.

As FIG. 13 shows, it was confirmed that:
DMTMM was a more efficient reagent for cross-linking the polymer than EDC/NHS.
Purification method was also more efficient as the control reaction with no cross-linking reagent revealed only peaks for sodium hyaluronate in NMR spectra after purification.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A hydrogel composition for use in a method of treating interstitial cystitis in a mammal, in which the composition comprises crosslinked high molecular weight hyaluronan particles dispersed and suspended throughout a hydrogel matrix, and in which the hydrogel composition is formulated for direct bladder instillation in a human.

2. A hydrogel composition of claim 1, in which the hydrogel composition is administered to the human by direct bladder instillation.

3. A hydrogel composition of claim 1, where the hydrogel composition provides a barrier effect to facilitate the restoration/regeneration of the urothelium layer of the bladder.

4. A hydrogel composition of claim 1, where the hydrogel composition suppresses inflammatory cytokine production.

5. A hydrogel composition of claim 1, in which where the hydrogel composition decreases the permeability of the bladder wall.

6. A hydrogel composition of claim 1, in which the hydrogel matrix is hyaluronan.

7. A hydrogel composition of claim 4, in which the hydrogel matrix is crosslinked high molecular weight hyaluronan.

8. A hydrogel composition according to claim 1, in which the HA particles are nano-sized particles, or agglomerates of nano-sized HA particles, or a combination thereof.

9. A hydrogel composition according to claim 1, in which a weight ratio of HA particles to hydrogel matrix is about 1:10 to 10:1.

10. A hydrogel composition according to claim 1, in which a weight ratio of HA particles to hydrogel matrix is about 1:2 to 2:1.

11. A hydrogel composition according to claim 1, in which the HA is chemically crosslinked with a crosslinking moiety.

12. A hydrogel composition according to claim 1, in which the crosslinking moiety is a functionalised ethylene glycol.

13. A hydrogel composition according to claim 1, in which the functionalised ethylene glycol is a PEG-amine.

14. A hydrogel composition according to claim 1, in which the HA is crosslinked with EDC/NHS or 4-(4,6-dimethoxy-1,3,5-triazin-2-yl)-4-methylmorpholinium chloride (DMTMM) chemistry.

15. A hydrogel composition according to claim 1, in which the composition is administered once weekly for 4 to 12 weeks.

16. A hydrogel composition according to claim 1, in which the composition is administered by a 8F or 10F catheter.

17. A hydrogel composition comprising crosslinked hyaluronan particles dispersed and suspended throughout a crosslinked hyaluronan hydrogel matrix, wherein the hyaluronan is high molecular weight hyaluronan, and in which the hydrogel is formulated for direct bladder instillation in a human.

18. A hydrogel composition according to claim 17 in which the HA particles are nano-sized particles, or agglomerates of nano-sized HA particles, or a combination thereof.

19. A hydrogel composition according to claim 18 in which the nano-sized HA particles have an average particle size of 100-900 nm.

20. A hydrogel composition according to claim 18 in which the nano-sized HA particles have an average particle size of 400-600 nm.

21.-62. (canceled)
